(19) United States
(12) Patent Application Publication
(10) Pub. No.: US 2022/0169912 A1
(43) Pub. Date: Jun. 2, 2022

(54) LOW-DOSAGE HYDRATE INHIBITORS

(71) Applicant: Halliburton Energy Services, Inc., Houston, TX (US)

(72) Inventors: Loan Vo, Houston, TX (US); Deepak S. Monteiro, Houston, TX (US)

(21) Appl. No.: 17/600,515

(22) PCT Filed: May 17, 2019
(86) PCT No.: PCT/US2019/032809

§ 371 Date: Sep. 30, 2021

(51) Int. Cl. C09K 8/52; C07C 233/36; C07C 69/96

(52) U.S. Cl. CPC C09K 8/52; C09K 2208/22; C07C 69/96; C07C 233/36

(57) ABSTRACT

Low-dosage hydrate inhibitor additives and methods of using such additives to, for example, inhibit the formation of gas hydrate agglomerates are provided. In some embodiments, introducing a low-dosage hydrate inhibitor additive into a fluid including at least one component selected from the group consisting of: water, a gas, a liquid hydrocarbon, and any combination thereof, wherein the low-dosage hydrate inhibitor additive includes at least one compound having the structural formula: wherein each of R¹, R², and R³ is independently a C₁ to C₆ hydrocarbon chain, wherein R⁴ is a C₁ to C₅₀ hydrocarbon chain, and wherein X′ is selected from the group consisting of wherein R⁵ is a methyl or ethyl group, and any combination thereof.

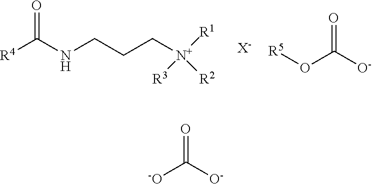

### BACKGROUND

The present disclosure relates to compositions and methods useful in processes involving fluid flowing through, or contained in, wellbores penetrating subterranean formations, vessels, or conduits, such as pipes used, e.g., for the production and/or transport of petroleum products, natural gas, and the like.

Gas hydrates are solids that may agglomerate in a fluid that is flowing or is substantially stationary, under certain temperature and pressure conditions. For example, gas hydrates may form during hydrocarbon production from a subterranean formation, in particular in pipelines and other equipment during production operations. Hydrates may impede or completely block flow of hydrocarbons or other fluid flowing through such pipelines. These blockages not only may decrease or stop production, potentially costing millions of dollars in lost production, but also may be very difficult and dangerous to mediate. Unless properly handled, gas hydrates may be volatile and/or explosive, potentially rupturing pipelines, damaging equipment, endangering workers, and/or causing environmental harm.

Gas hydrates may form when water molecules become bonded together after coming into contact with certain “guest” gas or liquid molecules. Hydrogen bonding causes the water molecules to form a regular lattice structure, like a cage, that is stabilized by the guest gas or liquid molecules entrapped within the lattice structure. The resulting crystalline structure may precipitate as a solid gas hydrate. Guest molecules can include any number of molecules such as, for example, carbon dioxide, methane, ethane, butane, propane, hydrogen, helium, freon, halogen, noble gases, and the like.

### BRIEF DESCRIPTION OF THE DRAWINGS

These drawings illustrate certain aspects of some of the embodiments of the present disclosure and should not be used to limit or define the claims.

FIG. 1 is a diagram illustrating a low-dosage hydrate inhibitor additive in accordance with certain embodiments of the present disclosure.

FIG. 2 is a diagram illustrating an example reaction process in accordance with certain embodiments of the present disclosure.

FIG. 3 is a diagram illustrating an injection system used in accordance with certain embodiments of the present disclosure.

While embodiments of this disclosure have been depicted, such embodiments do not imply a limitation on the disclosure, and no such limitation should be inferred. The subject matter disclosed is capable of considerable modification, alteration, and equivalents in form and function, as will occur to those skilled in the pertinent art and having the benefit of this disclosure. The depicted and described embodiments of this disclosure are examples only, and not exhaustive of the scope of the disclosure.

### DESCRIPTION OF CERTAIN EMBODIMENTS

Illustrative embodiments of the present disclosure are described in detail herein. In the interest of clarity, not all features of an actual implementation may be described in this specification. It will of course be appreciated that in the development of any such actual embodiment, numerous implementation-specific decisions may be made to achieve the specific implementation goals, which may vary from one implementation to another. Moreover, it will be appreciated that such a development effort might be complex and time-consuming, but would nevertheless be a routine undertaking for those of ordinary skill in the art having the benefit of the present disclosure.

To facilitate a better understanding of the present disclosure, the following examples of certain embodiments are given. In no way should the following examples be read to limit, or define, the scope of the invention. Embodiments of the present disclosure involving wellbores may be applicable to horizontal, vertical, deviated, or otherwise nonlinear wellbores in any type of subterranean formation. Embodiments may be applicable to any wells, including, but not limited to injection wells, monitoring wells, and production wells, including hydrocarbon or geothermal wells.

The present disclosure relates to compositions and methods useful in processes involving fluid flowing through, or contained in, subterranean formations, wellbores, vessels, conduits, such as pipes used, e.g., for the production and/or transport of petroleum products, natural gas, and the like. More particularly, the present disclosure relates to LDHI additives and method of using such LDHI additives to, for example, inhibit the formation of gas hydrate agglomerates.

In certain embodiments, the present disclosure may provide LDHI additives including a lipophilic tail, a hydrophilic head, and a linking moiety. In some embodiments, the LDHI additive may be provided, used, and/or introduced as a salt. In certain embodiments, the present disclosure further provides methods of using such LDHI additives to inhibit the formation of one or more hydrates in a fluid. For example, certain embodiments of the present disclosure provide methods of adding one or more LDHI additives of the present disclosure to a fluid including any one or more of water, a gas, a liquid hydrocarbon, and any combination thereof. In certain embodiments, such a method may include adding to the fluid an effective amount of a LDHI additive of the present disclosure to inhibit, retard, reduce, control, delay, and/or the like the formation of hydrate agglomerates.

Among the many advantages to the compositions and methods of the present disclosure, only some of which are alluded to herein, the LDHI additives and methods of the present disclosure may, among other benefits, provide for enhanced anti-agglomeration properties and/or enhanced inhibition, retardation, mitigation, reduction, control, delay, and/or the like of agglomeration of hydrates and/or hydrate-forming compounds. In certain embodiments, agglomeration of hydrates and/or hydrate-forming compounds may be inhibited (and the like) to a greater degree than that achieved using other hydrate inhibition means. In certain embodiments, a smaller quantity of the LDHI additives of the present disclosure may achieve a similar degree of inhibition of agglomeration of hydrates and/or hydrate-forming compounds as a greater amount of other LDHIs. In certain embodiments, the LDHI additives and methods of the present disclosure may be more effective in high-temperature environments than other LDHIs.

In certain embodiments, the LDHI additives of the present disclosure may at least partially inhibit, retard, reduce, control, and/or delay the agglomeration of hydrates and/or hydrate-forming compounds during and/or after exposure to high temperatures. In such embodiments, the LDHI additives of the present disclosure may not substantially degrade after an extended period of time at such high temperatures. As used herein, “substantially” and variations of that term may refer to a majority of, or mostly, as in at least about 50%, 60%, 70%, 80%, 90%, 95%, 96%, 97%, 98%, 99%, 99.5%, 99.9%, 99.99%, or at least about 99.999% or more. In certain embodiments, the LDHI additives of the present disclosure may be substantially or completely free of halogens, which may allow for processing of fluids, such as refining, including the LDHI additives of the present disclosure in facilities without the need to remove halogens from the fluids. Additionally, it is believed that the LDHI additives of the present disclosure may provide benefits and/or may be used as an additive for purposes other than hydrate inhibition, such as, for example, corrosion inhibition.

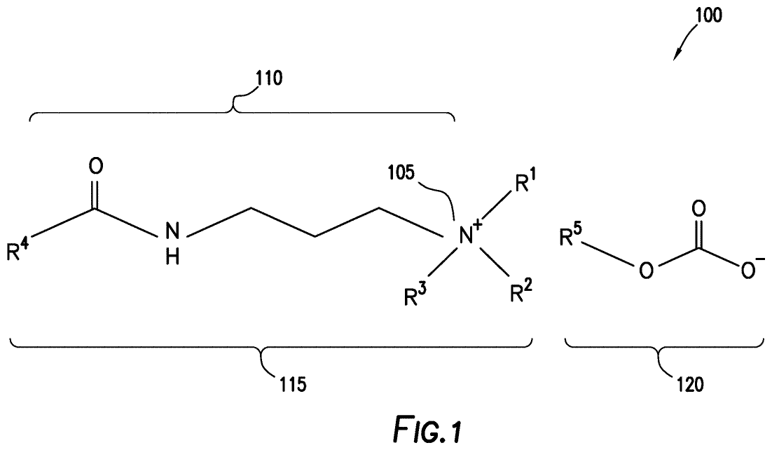

The LDHI additives of the present disclosure may include a hydrophilic head including a cation moiety that may be a quaternary ammonium cation moiety or a tertiary ammonium cation moiety. FIG. 1 illustrates the chemical structure for certain LDHI additives of the present disclosure. In certain embodiments, the cation moiety in the LDHI additives of the present disclosure may be bonded to other moieties of the LDHI additive, for example, as shown with respect to the hydrophilic head 105 of the LDHI additive 100 in FIG. 1. In certain embodiments, the cation moiety may be substantially of the composition—R¹R²R³N⁺—. Each of R¹, R², and R³ may independently include either a hydrogen atom or a C₁ to C₆ hydrocarbon chain. As used herein, a “hydrocarbon chain” may, unless otherwise specifically noted, be branched, unbranched, non-cyclic, and/or cyclic; it may be substituted or unsubstituted (that is, it may or may not contain one or more additional moieties or functional groups in place of one or more hydrogen atoms in the hydrocarbon chain); and/or it may be saturated or unsaturated. Furthermore, as used herein, the nomenclature “Cₓ to C_{y}” refers to the number of carbon atoms in the hydrocarbon chain (here, ranging from x to y carbon atoms).

In certain embodiments, R¹, R², and/or R³ may be a hydrogen atom. In certain embodiments, only one of R¹, R², and R³ may be a hydrogen atom. In those embodiments, the cation moiety is a tertiary ammonium cation moiety. In such embodiments wherein R¹, R², and/or R³ includes a C₁ to C₆ hydrocarbon chain, the hydrocarbon chain may include any one or more hydrocarbon groups selected from the group consisting of: alkyl, alkenyl, alkynyl, aryl, arylalkyl, arylalkenyl, alkylaryl, alkenylaryl, and any combination thereof. In such embodiments, any one or more of R¹, R², and R³ may be branched, unbranched, non-cyclic, cyclic, saturated, and/or unsaturated. In certain embodiments, each of R¹, R², and R³ may independently include (i) as few as any one of: 1, 2, 3, 4, 5, and 6 carbon atoms, and (ii) as many as one of: 4, 5, and 6 carbon atoms. For example, suitable ranges of numbers of carbon atoms in each of R¹, R², and R³ according to various embodiments of the present disclosure include, but are not limited to, 1 to 2, 1 to 3, 1 to 4, 1 to 5, 1 to 6, 2 to 4, 3 to 5, and 4 to 6, and the like.

In some embodiments, any one or more of R¹, R², and R³ may include a C₁ to C₆ alkyl chain. In some embodiments, any one or more of R¹, R², and R³ may include a C₂ to C₆ alkenyl or alkynyl chain (in which case at least 2 carbon atoms are necessary to form an alkenyl or alkynyl chain). In some embodiments, any one or more of R¹, R², and R³ may include a C₃ to C₆ cyclic moiety (in which case at least 3 carbon atoms are necessary to form a cyclic moiety). In certain embodiments, any one or more of R¹, R², and R³ may be substituted (e.g., it may include any one or more functional groups in addition to the hydrocarbon groups described above), so long as the cation moiety remains hydrophilic.

The LDHI additives of the present disclosure may further include a lipophilic tail. For example, as shown in FIG. 1, the LDHI additive 100 includes a lipophilic tail R⁴. In certain embodiments, the lipophilic tail of the LDHI additives of the present disclosure may include a C₁ to C₅₀ hydrocarbon chain. In certain embodiments, the hydrocarbon chain on the lipophilic tail may be branched or unbranched, cyclic or non-cyclic, saturated or saturated, and/or may be any one or more of alkyl, alkenyl, alkynyl, and aryl groups, and/or any combination thereof. In certain embodiments, the lipophilic tail may further optionally be substituted with any one or more functional groups, so long as such substituted functional group(s) do not alter the lipophilic and/or hydrophobic nature of the lipophilic tail. In certain embodiments, the lipophilic tail may include (i) as few as any one of: 1, 2, 3, 4, 5, 6, 7, 8, 9, 10, 11, 12, 13, 14, 15, 16, 17, 18, 19, and 20 carbon atoms, and (ii) as many as any one of: 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, and 50 carbon atoms. For example, suitable ranges of numbers of carbon atoms in the lipophilic tail according to various embodiments of the present disclosure include, but are not limited to, 1 to 5, 3 to 5, 4 to 8, 5 to 15, 8 to 18, 12 to 16, 8 to 20, 10 to 20, 15 to 20, and the like. It will be appreciated by one of ordinary skill in the art having the benefit of the present disclosure that even in such embodiments, additional lipophilic tails could be included in the LDHI additive (e.g., at a point along the backbone 115 of the LDHI additive 100).

The LDHI additives of the present disclosure may further include a linking moiety. As used herein, “linking moiety” refers to any portion of the LDHI additive that provides spacing between the hydrophilic head and the lipophilic tail. In certain embodiments, the lipophilic tail may be connected to the hydrophilic head via the linking moiety. For example, in the LDHI additive 100 shown in FIG. 1 the lipophilic tail R⁴ is connected to the hydrophilic head 105 via the linking moiety 110. In certain embodiments, the linking moiety may provide sufficient spacing so that the LDHI additive maintains an overall substantially amphiphilic character.

In certain embodiments, the linking moiety may include any length hydrocarbon chain, branched or unbranched, and/or saturated or unsaturated (so long as the overall LDHI additive maintains amphiphilic character). Hydrocarbon chain lengths include C₁ to C₅₀ chains or longer. In certain embodiments, the linking moiety may be any one or more of methyl, ethyl, propyl, butyl, pentyl, hexyl, heptyl, octyl, nonyl, decyl, etc. In certain embodiments, the linking moiety may be substituted such that it includes any kind and/or any number of functional groups (so long as the LDHI additive maintains both hydrophobic and hydrophilic portions). In such embodiments, the one or more functional groups that may be included on the linking moiety according to some embodiments should not adversely affect the hydrophilic nature of a hydrophilic head, nor should they adversely affect the lipophilic nature of a lipophilic tail. Examples of suitable functional groups that may be included in the linking moieties, the lipophilic tails, and/or the R-groups (R¹, R², R³) of the present disclosure may include any one or more of: an ester, ether, amine, sulfonamide, amide, ketone, carbonyl, isocyanate, urea, urethane, and any combination thereof. In some embodiments, the one or more functional groups on the linking moiety may include any group capable of reacting with an amine (so long as that functional group's inclusion in the linking moiety allows the LDHI additive to maintain amphiphilic character). The LDHI additive 100 of FIG. 1 includes an example of a linking moiety 110 including an amide as well as a propyl group.

The LDHI additives of the present disclosure may instead or in addition be characterized as reaction products. For instance, in some embodiments, the present disclosure provides LDHI additives that may be characterized as the reaction product of: (1) dialkylaminopropylamine and (2) one or more fatty acids or fatty acid esters. In such embodiments, the two dialkyl groups of 5 the dialkylaminopropylamine may be either the same or different, and the R¹ and R² groups of the cation moiety may depend upon, among other factors, the identity of the two dialkyl group(s). In some embodiments, the reaction product of (1) dialkylaminopropylamine and (2) one or more fatty acids or fatty acid esters may further be reacted with (3) an alkyl carbonate or dialkyl carbate. In such embodiments, R³ of the cation moiety may depend upon the alkyl group of the alkyl carbonate 10 or dialkyl carbate. In certain embodiments, the composition of the lipophilic tail of the LDHI additive may depend upon the fatty acid(s) and/or fatty acid ester(s) used as reactant(s). In certain embodiments, the fatty acid and/or fatty acid ester may include one or more functional groups and a portion of the functional group may be included in the linking moiety of the resultant reactant product. Suitable fatty acids and/or fatty acid esters for reaction may include a saturated fatty acid and/or an unsaturated fatty acid, such as one or more selected from the group consisting of: corn oil, canola oil, coconut oil, safflower oil, sesame oil, palm oil, tall oil, cottonseed oil, soybean oil, olive oil, sunflower oil, hemp oil, wheat germ oil, palm kernel oil, vegetable oil, caprylic acid, capric acid, lauric acid, stearic acid, myristic acid, myristoleic acid, palmitic acid, palmitoleic acid, stearic acid, sapienic acid, elaidic acid, vaccenic acid, linoleic acid, arachidic acid, arachidonic acid, eicosapentaenoic acid, erucic acid, docosahexaenoic acid, behenic acid, lignoceric acid, cerotic acid, oleic acids (cis- and trans-), any fatty acid derived therefrom, and any combination thereof.

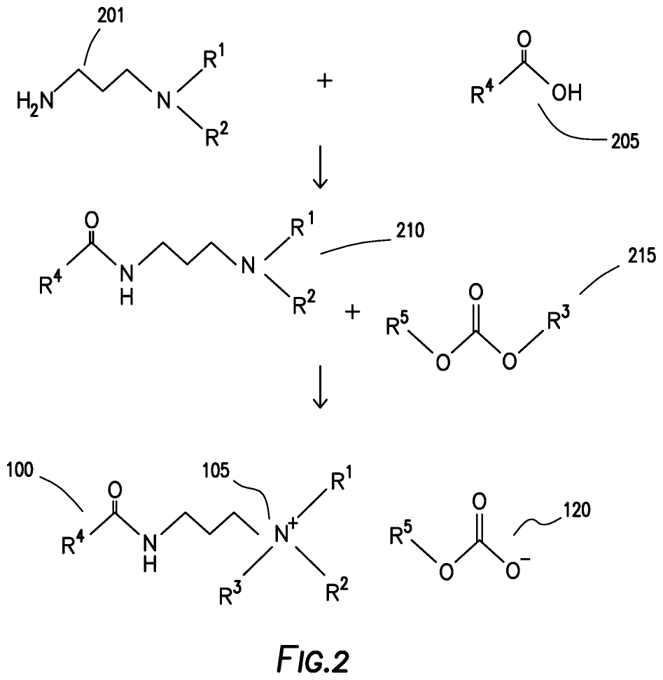

FIG. 2 illustrates a potential reaction scheme for forming a LDHI additive in accordance with certain embodiments of the present disclosure. In the reaction scheme shown, dialkylaminopropylamine 201 (which, as shown in FIG. 2, includes hydrocarbon chains R¹ and R²) reacts with fatty acid ester 205 (which, as shown in FIG. 2, includes hydrocarbon chain R⁴), forming amide intermediate 210. In certain embodiments, this reaction may occur at about 165° C. to about 170° C. for about 10 hours. Amide intermediate 210 in turn reacts with alkyl carbonate 215 (which, as shown in FIG. 2, includes hydrocarbon chain R³ and methyl or ethyl group R⁵) to form LDHI additive 100. In some embodiments, R³ may be a methyl or ethyl group. In certain embodiments, the alkyl carbonate 215 is a dialkyl carbonate. In some embodiments, the reaction between the amide intermediate 210 and the alkyl carbonate may occur at around 121° C., around 400 psi, and in the presence of nitrogen gas. LDHI additive 100 includes a lipophilic tail R⁴ (retaining the hydrocarbon structure R⁴ of the fatty acid ester 205) and a hydrophilic head 105 including a R-groups R¹ and R² (retaining the hydrocarbon structure R¹ and R² of the dialkylaminopropylamine 201) and R³ (retaining the hydrocarbon structure R³ of the alkyl carbonate 215). Such reactions may in some embodiments take place at about 80° C. to about 250° C. 5 at approximately atmospheric pressure or lower pressure. It will be appreciated by one of ordinary skill in the art having the benefit of the present disclosure that various modifications may be made to this reaction scheme to produce other embodiments. Furthermore, in yet other embodiments, another reactant besides fatty acids may be used. Examples of suitable other reactants include, but are not limited to, esters, sulfonamides, amides, ketones, carbonyls, isocyanates, urea, urethane, and any combination thereof.

In certain embodiments, the LDHI additives of the present disclosure may be provided, used, and/or introduced as a salt of one or more of the compounds described herein. In such embodiments, the salt may include a counter anion. For example, the LDHI additive 100 as shown in FIGS. 1 and 2 include a salt with a carbonate counter anion 120. In certain embodiments, such salts may wholly or partially dissociate in aqueous solution. In other embodiments, the salts may remain substantially associated (either with the original anion or with other ions from solution). It will be appreciated by one of ordinary skill in the art having the benefit of this disclosure that salts may be formed with other counter anions instead of or in addition to carbonate counter anions. Suitable counter anions may include, for example, any one or more of hydroxide, carboxylate, halide, sulfate, organic carbonate, and any combination thereof. In certain embodiments, such counter anions may include an alkyl group. For example, the counter anion 120 of FIG. 1 includes R⁵, which may include a methyl or ethyl group.

In certain embodiments, the LDHI additives of the present disclosure may have substantially the following structural formula:

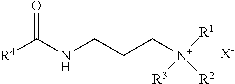

In such embodiments, each of R¹ and R² may independently be a C₁ to C₆ hydrocarbon chain according to the previous discussion of the R¹ and R² groups; R³ may be selected from the group consisting of hydrogen and a C₁ to C₆ hydrocarbon chain according to the previous discussion of the R³ group; R⁴ may be a C₁ to C₅₀ hydrocarbon chain according to the previous discussion of the R⁴ group. In some embodiments, R³ is a methyl or ethyl group. In certain embodiments, X⁻ is an anion selected from the group consisting of:

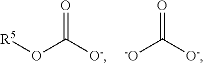

and any combination thereof. In certain embodiments, R⁵ may be a methyl or ethyl group.

The present disclosure in certain embodiments further provides methods of using the LDHI additives of the present disclosure. In certain embodiments, the LDHI additives of the present disclosure may be used to inhibit, retard, mitigate, reduce, control, and/or delay the formation of one or more hydrates or agglomerates of hydrates. In certain embodiments, one or more LDHI additives of the present disclosure may be introduced into a fluid including any one or more of water, a gas, a liquid hydrocarbon, and any combination thereof. Although listed separately from liquid hydrocarbon, the gas may in some embodiments include gaseous hydrocarbon, though the gas need not necessarily include hydrocarbon. In certain embodiments, the fluid includes water with less than 120,000 ppm total dissolved solids. In some embodiments, the fluid includes water with less than 6,000 ppm total dissolved solids. In certain embodiments, the LDHI additive may be introduced into the fluid through a conduit or an injection point. In certain embodiments, one or more LDHI additives of the present disclosure may be introduced into a wellbore, a conduit, a vessel, and the like and may contact and/or be introduced into a fluid residing therein.

In certain embodiments, the fluid may be flowing or it may be substantially stationary. The fluid may be within a vessel, or within a conduit (e.g., a conduit that may transport the fluid), or within a subterranean formation and/or a wellbore penetrating a portion of the subterranean formation. Examples of conduits include, but are not limited to, pipelines, production piping, subsea tubulars, process equipment, and the like as used in industrial settings and/or as used in the production of oil and/or gas from a subterranean formation, and the like. The conduit may in certain embodiments penetrate at least a portion of a subterranean formation, as in the case of an oil and/or gas well. In particular embodiments, the conduit may be a wellbore or may be located within a wellbore penetrating at least a portion of a subterranean formation. Such oil and/or gas well may, for example, be a subsea well (e.g., with the subterranean formation being located below the sea floor), or it may be a surface well (e.g., with the subterranean formation being located belowground). A vessel or conduit according to other embodiments may be located in an industrial setting such as a refinery (e.g., separation vessels, dehydration units, pipelines, heat exchangers, and the like), or it may be a transportation pipeline.

In some embodiments, the LDHI additives of the present disclosure initially may be incorporated into a composition prior to being introduced into the fluid, conduit, vessel, or formation. The composition may be any suitable composition in which the LDHI additive may be included. For example, in some embodiments, the composition may be a treatment fluid for use in a wellbore penetrating a subterranean formation during, for instance, oil and/or gas recovery operations. The composition may include a solvent for the LDHI additive. Suitable solvents include any one or more of: toluene, xylene, methanol, isopropyl alcohol, any alcohol, glycol, any organic solvent, and any combination thereof.

In certain embodiments, one or more LDHI additives of the present disclosure may be introduced into and/or contact the fluid in an amount from about 0.1% to about 5.5% by volume based on the volume of water in the fluid (or in other words, about 0.1% to about 5.5% by volume based on water cut). In various embodiments, an effective amount of LDHI additive for inhibiting, 15 retarding, mitigating, reducing, controlling, delaying, and/or the like agglomeration of hydrates may be as low as any of: 0.1, 0.25, 0.50, 0.75, 1.00, 1.25, 1.50, 1.75, 2.00, 2.25, and 2.50% by volume based on water cut. An effective amount may be as high as any of: 0.50, 0.75, 1.0, 1.25, 1.50, 1.75, 2.0, 2.25, 2.50, 2.75, 3.0, 3.25, 3.50, 3.75, 4.0, 4.50, 5.0, and 5.50% by volume based on water cut. Thus, in some embodiments, an effective amount of LDHI additives of the present disclosure for inhibiting, retarding, mitigating, reducing, controlling, delaying, and/or the like agglomeration of hydrates may be about 0.1% to about 3% volume based on water cut of the fluid; in other embodiments, about 0.1% to about 2% volume based on water cut of the fluid; in other embodiments, about 0.25% to about 1.5% volume based on water cut of the fluid; and in other embodiments, about 0.5% to about 1.0% volume based on water cut of the fluid.

In certain embodiments, one or more LDHI additives of the present disclosure may be introduced to and/or contact any of various fluids having different water cuts (i.e., the ratio of the volume of water in the fluid to the total volume of the fluid). For example, in some embodiments the water cut of the fluid may be about 1 to about 65%. In other embodiments, the water cut may be as low as any one of: 1, 5, 10, 15, 20, 25, 30, 35, 40, 45, 50, 55, 60, and 65%; while the water cut may be as high as any one of: 5, 10, 15, 20, 25, 30, 35, 40, 45, 50, 55, 60, 65, 70, 75, 80, 85, 90, and 95%. In certain embodiments, a fluid may have a water cut of 5% or more, 10% or more, 15% or more, 20% or more, 30% or more, 40% or more, 50% or more, or 60% or more, up to about 99%. In yet other embodiments, one or more LDHI additives of the present disclosure may be introduced into or contact a fluid with any water cut ranging from about 1% to about 99%.

In certain embodiments, the fluid to which one or more LDHI additives of the present disclosure may be introduced optionally may include any number of additional additives. Examples of such additional additives include, but are not limited to, salts, surfactants, acids, proppant particulates, diverting agents, fluid loss control additives, nitrogen, carbon dioxide, surface modifying agents, tackifying agents, foamers, corrosion inhibitors, scale inhibitors, catalysts, clay control agents, biocides, friction reducers, antifoam agents, bridging agents, flocculants, H₂S scavengers, C₀₂ scavengers, oxygen scavengers, lubricants, viscosifiers, breakers, weighting agents, relative permeability modifiers, resins, wetting agents, coating enhancement agents, filter cake removal agents, antifreeze agents (e.g., ethylene glycol), and the like. A person skilled in the art, with the benefit of this disclosure, will recognize the types of additives that may be included in the fluids of the present disclosure for a particular application. It further will be appreciated by one of ordinary skill in the art having the benefit of the present disclosure that the amount of the LDHI additives of the present disclosure effective for inhibiting, retarding, reducing, controlling, delaying, and/or the like hydrates may depend upon, for example, the volume of water in the fluid and/or additives in the fluid.

In certain embodiments, the LDHI additives of the present disclosure may be exposed to and may remain substantially stable at) higher than ambient temperatures. For example, in some embodiments, the compositions of the present disclosure may be exposed to a temperature above about 200° F. In certain embodiments, LDHI additives of the present disclosure may be exposed to a temperature from about 200° F. to about 400° F. In some embodiments, the LDHI additives of the present disclosure may be exposed to a temperature from about 200° F. to about 250° F., in other embodiments, from about 250° F. to about 300° F., in other embodiments, from about 300° F. to about 350° F., and in other embodiments, from about 350° F. to about 400° F. In some embodiments, the LDHI additives of the present disclosure may be exposed to a temperature from about 250° F. to about 275° F., in other embodiments, from 275° F. to about 300° F., in other embodiments, from about 300° F. to about 325° F., and in other embodiments from about 325° F. to about 350° F.

In certain embodiments, the LDHI additives may be exposed to a temperature of above 30 about 200° F. when introduced into or contacting a fluid having a temperature of above about 200° F. In such embodiments, the fluid may have a temperature from about 200° F. to about 400° F. In some embodiments, the fluid may have a temperature from about 250° F. to about 350° F. In certain embodiments, the LDHI additive may be exposed to a temperature above about 200° F. in a conduit, an injection point, a wellbore, and the like having a temperature above about 200° F. through which the LDHI additive travels when being introduced into or contacting the fluid.

In certain embodiments, the LDHI additive of the present disclosure may be exposed to lower than ambient temperatures. For example, in some embodiments, when introduced into an umbilical, the LDHI additive of the present disclosure may be exposed to ambient temperatures or temperatures at or above about 100° F.

In certain embodiments, the LDHI additives of the present disclosure may be exposed to a temperature above about 200° F. for an extended period of time without substantially degrading. In certain embodiments, the LDHI additives of the present disclose may remain in a fluid having a temperature above 200° F. for an extended period of time without substantially degrading. In some embodiments, the LDHI additives of the present disclosure may be exposed to a temperature above about 200° F., alternatively above about 250° F., alternatively above 300° F., alternatively above about 350° F., or alternatively above about 400° F. for an extended period of time without substantially degrading. In some embodiments, the LDHI additives of the present disclosure may be exposed to a temperature above about 200° F. without substantially degrading for up to about: 1, 2, 3, 4, 5, 6, 7 or more days. In certain embodiments, the LDHI additives of the present disclosure do not substantially degrade after about 7 days at a temperature above about 200° F.

In certain embodiments, the LDHI additives of the present disclosure may be introduced into a wellbore, subterranean formation, vessel, and/or conduit (and/or into a fluid within any of the foregoing) using any method or equipment known in the art. For example, the LDHI additives of the present disclosure may be applied to a subterranean formation and/or wellbore using batch treatments, squeeze treatments, continuous treatments, and/or any combination thereof. In certain embodiments, a batch treatment may be performed in a subterranean formation by stopping production from the well and pumping the dissolved hydrate inhibitors into a wellbore, which may be performed at one or more points in time during the life of a well. In other embodiments, a squeeze treatment may be performed by dissolving a LDHI additive of the present disclosure in a suitable solvent at a suitable concentration and squeezing that solvent carrying the hydrate inhibitor downhole into the formation, allowing production out of the formation to bring the hydrate inhibitor to its desired location. In other embodiments, a LDHI additive of the present disclosure may be injected into a portion of a subterranean formation using an annular space or capillary injection system to continuously introduce the LDHI additive into the formation. In certain embodiments, a composition (such as a treatment fluid) including a LDHI additive of the present disclosure may be circulated in the wellbore using the same types of pumping systems and equipment at the surface that are used to introduce treatment fluids or additives into a wellbore penetrating at least a portion of the subterranean formation.

In certain embodiments, the methods of the present disclosure include applying the LDHI additive to a fluid. In some embodiments, the method of applying the LDHI additive to prevent hydrate plugging includes introducing an LDHI additive into an umbilical line or a capillary line in which a fluid is located.

In certain embodiments, the fluids or additives may be formed at a well site where the operation or treatment is conducted, either by batch mixing or continuous (“on-the-fly”) mixing. The term “on-the-fly” is used herein to include methods of combining two or more components wherein a flowing stream of one element is continuously introduced into a flowing stream of at least one other component so that the streams are combined and mixed while continuing to flow as a single stream as part of the on-going treatment. Such mixing can also be described as “real-time” mixing. In other embodiments, the treatment fluids of the present disclosure may be prepared, either in whole or in part, at an offsite location and transported to the site where the treatment or operation is conducted. In introducing a composition of the present disclosure into a vessel, conduit (e.g., an umbilical, capillary, or tubing), wellbore, portion of a subterranean formation, components of the composition may be mixed together at the surface and introduced into the vessel, conduit, wellbore, and/or formation together, or one or more components may be introduced into the vessel, conduit, wellbore, and/or formation at the surface separately from other components such that the components mix or intermingle in a portion of the vessel, conduit, wellbore, and/or formation to form a composition. In either such case, the composition is deemed to be introduced into at least a portion of the vessel, conduit, wellbore, and/or subterranean formation for purposes of the present disclosure.

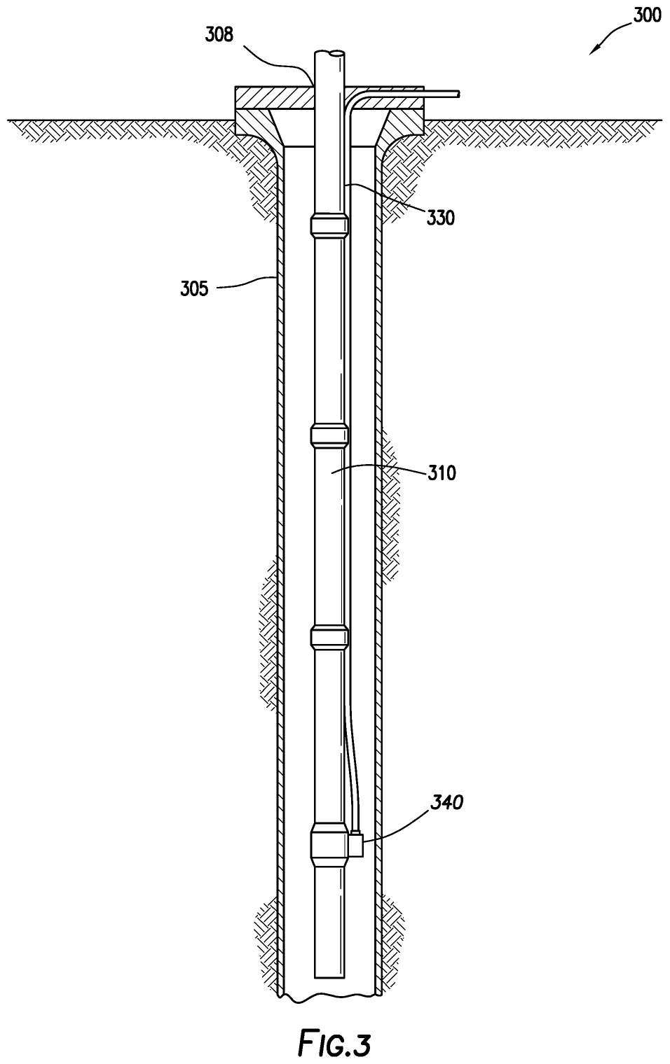

For example, a LDHI additive of the present disclosure may be introduced into a wellbore and/or tubing using a capillary injection system as shown in FIG. 3. Referring now to FIG. 3, wellbore 305 has been drilled to penetrate a portion of a subterranean formation 300. A tubing 310 (e.g., production tubing) has been placed in the wellbore 305. A capillary injection tube 330 is disposed in the annular space between the outer surface of tubing 310 and the inner wall of wellbore 305. The capillary injection tube 330 is connected to a side-pocket mandrel 340 at a lower section of the tubing 310. A LDHI additive of the present disclosure may be injected into capillary injection tube 330 at the wellhead 308 at the surface such that it mixes with production fluid at or near the side-pocket mandrel 340. As the production fluid flows through the tubing 310, the LDHI additive may prevent, inhibit, retard, reduce, control, and/or delay the formation of one or more hydrates within the tubing 310. Other capillary injection systems and side pocket mandrel devices (e.g., those used in gas lift production) may be used in a similar manner to the system shown in FIG. 3.

In certain embodiments, a LDHI additive of the present disclosure may be added to a conduit such as a pipeline where one or more fluids enter the conduit and/or at one or more other locations along the length of the conduit. In such embodiments, the LDHI additive may be added in batches or injected substantially continuously while the pipeline is being used.

Once introduced into a fluid, subterranean formation, wellbore, pipeline, or other location, the LDHI additive may inhibit, retard, reduce, control, and/or delay the formation of one or more hydrates or the agglomeration of hydrate crystals within the fluid, subterranean formation, wellbore, pipeline, or other location.

To facilitate a better understanding of the present disclosure, the following examples of certain aspects of certain embodiments are given. The following examples are not the only examples that could be given according to the present disclosure and are not intended to limit the scope of the disclosure or claims.

### EXAMPLE

Rocking cell tests were carried out on numerous samples of different compounds having structures according to some embodiments of the present disclosure. The rocking cell tests involved the injection of oil, water, and LDHI compound into a cell at representative conditions. Gas was injected into the cell to reach the desired working pressure. Each cell had a fixed volume and contained constant mass during the experiment; that is, oil, water, LDHI compound, and (in some cases) gas were injected at the beginning of the experiment, but thereafter the cell was closed to mass transfer in or out of the cell. Each cell also included a magnetic ball in the space where fluids are injected. The ball aided in agitation of the fluids during rocking. Magnetic sensors on both ends of the cell detected whether the magnetic ball's movements through the fluids were hindered during rocking, thereby indicating the presence of hydrates. The cell also permitted visual observation of its contents for formation of hydrates during the experiment.

The oil used for these tests was dodecane and the water phase were between 3.5-6% by weight NaCl brines. Specific oils, water cuts, salinities, and LDHI dosages for each test are shown below. The oil was pre-conditioned by heating and shaking at 70° C. for 1 hour. The proper amount of oil, water and inhibitor were injected into the cells per the values listed in Tables 2-4 below. Thereafter, the cells were pressurized to between 2,000 psi and 2,800 psi with Green Canyon gas, a common Gulf of Mexico Type II hydrate former. The composition of Green Canyon gas, used for this study, is provided in Table 1.

TABLE 1
Composition of Green Canyon Gas

| Composition | Mole % |
|---|---|
| N2 | 0.39 |
| nC1 | 87.26 |
| nC2 | 7.57 |
| nC3 | 3.10 |
| iC4 | 0.49 |
| nC4 | 0.79 |
| iC5 | 0.20 |
| nC5 | 0.20 |

During the initial phase of each test, the cells were rocked at the prescribed angle and rate for a period of 2 hours in order to sufficiently emulsify the fluids and saturate the liquid phase with gas such that no further gas would be consumed by the liquid phase. Thereafter, the gas inlet valves were closed and the temperature was ramped down from 20° C. to 4° C. over 1 to 2 hours. After reaching the designated temperature, the cells were rocked at 15 cycles per minute and a 250 angle for around 18 hours. The fluids in the cells had water cuts of about 15%. Thereafter, the motor was programmed to stop for 6 hours with the cells horizontal to simulate a system shut in. The shut-in period lasted for at least 6 hours, varying only so that the restart could be visually observed. Observations were made throughout the tests. Particular attention was paid to hydrate formation during the period before shut-in and the restart.

The performance of each hydrate inhibitor was ranked as a “Pass” or a “Fail” based on visual observation and sensor data. When hydrate blockages impeded the motion of the ball, the cell was ranked as a “Fail.” If a cell visually passed, the sensors must also have showed no obstruction or hindrance to the movement of the ball for the cell to rank as a “Pass.”

Samples were prepared including LDHI additives of the present disclosure. The LDHI additives had the following base structure:

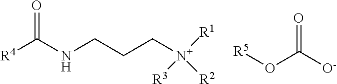

The conditions and the results for each test are shown below in Table 2.

TABLE 2
LDHI Additives Rocking Cell Test Results

| | | | | | New LDHI dosage | |
|---|---|---|---|---|---|---|
| Initial Pressure | Cooldown | | | Water | (v/v based on water | Overall |
| (psi) | time (hr.) | Oil | Salinity | Cut | cut) | result |
| 2800 | 1 | Dodecane | 6% | 35% | 2% | Pass |
| 2800 | 1 | Dodecane | 6% | 40% | 2% | Pass |
| 2000 | 2 | Dodecane | 3.5% | 35% | 2% | Pass |
| 2800 | 1 | Mission | 6% | 50% | 2.4% | Pass |
| | | Condensate | | | | |
| 2800 | 1 | Mission | 6% | 55% | 2.4% | Pass |
| | | Condensate | | | | |
| 2800 | 1 | ST220 | 6% | 50% | 2.4% | Pass |
| | | Condensate | | | | |
| 2800 | 1 | ST220 | 6% | 55% | 2.4% | Pass |
| | | Condensate | | | | |
| 2800 | 1 | Longhorn | 6% | 55% | 2.4% | Pass |
| 2800 | 1 | Longhorn | 6% | 60% | 2.4% | Pass |

As also indicated in Table 2, each LDHI additive was applied at the indicated dosage (2.0% or 2.4% v/v based on water cut) to fluids having different water cuts. As shown by the results in Table 2, the LDHI additive of the present disclosure passed under all of the tested conditions. This example demonstrates that the compositions and methods of the present disclosure may facilitate, among other benefits, the inhibition, retardation, reduction, control, and/or delay of agglomeration of hydrates and/or hydrate-forming compounds, including in fluids having a water cut of about 35% or greater.

An embodiment of the present disclosure is a method including introducing a low-dosage hydrate inhibitor additive into a fluid including at least one component selected from the group consisting of: water, a gas, a liquid hydrocarbon, and any combination thereof, wherein the low-dosage hydrate inhibitor additive includes at least one compound having the structural formula:

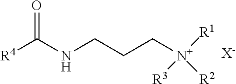

wherein each of R¹, R², and R³ is independently a C₁ to C₆ hydrocarbon chain, wherein R⁴ is a C₁ to C₅₀ hydrocarbon chain, and wherein X⁻ is selected from the group consisting of:

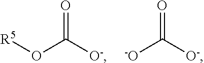

and any combination thereof, wherein R⁵ is a methyl or ethyl group.

In one or more embodiments described above, the low-dosage hydrate inhibitor additive is introduced into the fluid through an umbilical or a capillary line. In one or more embodiments described above, the low-dosage hydrate inhibitor additive does not substantially degrade for up to about 7 days. In one or more embodiments described above, the fluid resides within a location selected from the group consisting of: a conduit, a wellbore, a subterranean formation, and a vessel. In one or more embodiments described above, R³ is a methyl or ethyl group. In one or more embodiments described above, the fluid includes water and has a water cut of about 50% or greater. In one or more embodiments described above, the low-dosage hydrate inhibitor additive is introduced in an amount such that the low-dosage hydrate inhibitor additive is present in the fluid in an amount from about 0.1% to about 10% volume based on a water cut of the fluid. In one or more embodiments described above, the water is selected from the group consisting of: brine, deionized water, and any combination thereof.

In another embodiment, the present disclosure provides a method including introducing a low-dosage hydrate inhibitor additive into a wellhead of a wellbore penetrating at least a portion of a subterranean formation, wherein the low-dosage hydrate inhibitor additive includes at least one compound having the structural formula:

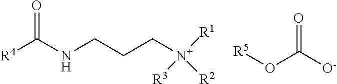

wherein each of R¹, R², and R³ is independently a C₁ to C₆ hydrocarbon chain, wherein R⁴ is a C₁ to C₅₀ hydrocarbon chain, wherein R⁴ is a C₁ to C₅₀ hydrocarbon chain, and wherein R⁵ is a methyl or ethyl group; and allowing the low-dosage hydrate inhibitor additive to contact a fluid in the wellbore.

In one or more embodiments described above, the fluid includes at least one component selected from the group consisting of: water, a gas, a liquid hydrocarbon, and any combination thereof. In one or more embodiments described above, fluid includes water and has a water cut of about 50% or greater. In one or more embodiments described above, the water is selected from the group consisting of: brine, deionized water, and any combination thereof. In one or more embodiments described above, the wellbore has a temperature from about 200° F. to about 350° F. In one or more embodiments described above, the low-dosage hydrate inhibitor additive does not substantially degrade after about 7 days. In one or more embodiments described above, the low-dosage hydrate inhibitor additive is introduced in an amount such that the low-dosage hydrate inhibitor additive is present in the fluid in an amount from about 0.1% to about 10% volume based on a water cut of the fluid.

In another embodiment, the present disclosure provides a method including introducing a low-dosage hydrate inhibitor additive into a conduit containing a fluid, wherein the low-dosage hydrate inhibitor additive includes at least one compound having the structural formula:

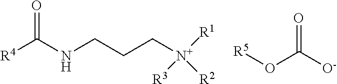

wherein each of R¹, R², and R³ is independently a C₁ to C₆ hydrocarbon chain, wherein R⁴ is a C₁ to C₅₀ hydrocarbon chain, and wherein R⁵ is a methyl or ethyl group.

In one or more embodiments described above, the fluid includes at least one component selected from the group consisting of: water, a gas, a liquid hydrocarbon, and any combination thereof. In one or more embodiments described above, the low-dosage hydrate inhibitor additive is introduced in an amount such that the low-dosage hydrate inhibitor additive is present in the fluid in an amount from about 0.1% to about 10% volume based on a water cut of the fluid. In one or more embodiments described above, the conduit includes a pipeline. In one or more embodiments described above, the fluid includes water and has a water cut of about 50% or greater.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. While numerous changes may be made by those skilled in the art, such changes are encompassed within the spirit of the subject matter defined by the appended claims. Furthermore, no limitations are intended to the details of construction or design herein shown, other than as described in the claims below. It is therefore evident that the particular illustrative embodiments disclosed above may be altered or modified and all such variations are considered within the scope and spirit of the present disclosure. In particular, every range of values (e.g., “from about a to about b,” or, equivalently, “from approximately a to b,” or, equivalently, “from approximately a-b”) disclosed herein is to be understood as referring to the power set (the set of all subsets) of the respective range of values. The terms in the claims have their plain, ordinary meaning unless otherwise explicitly and clearly defined by the patentee.

What is claimed is:

1. A method comprising:
introducing a low-dosage hydrate inhibitor additive into a fluid comprising at least one component selected from the group consisting of: water, a gas, a liquid hydrocarbon, and any combination thereof, wherein the low-dosage hydrate inhibitor additive comprises at least one compound having the structural formula: 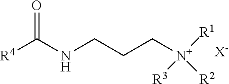
wherein each of R¹, R², and R³ is independently a C₁ to C₆ hydrocarbon chain,
wherein R⁴ is a C₁ to C₅₀ hydrocarbon chain, and
wherein X⁻ is selected from the group consisting of: 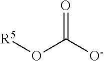
wherein R⁵ is a methyl or ethyl group, 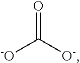
and any combination thereof.

2. The method of claim 1 wherein the low-dosage hydrate inhibitor additive is introduced into the fluid through an umbilical or a capillary line.

3. The method of claim 1 wherein the low-dosage hydrate inhibitor additive does not substantially degrade for up to about 7 days.

4. The method of claim 1 wherein the fluid resides within a location selected from the group consisting of: a conduit, a wellbore, a subterranean formation, and a vessel.

5. The method of claim 1 wherein R³ is a methyl or ethyl group.

6. The method of claim 1 wherein the fluid comprises water and has a water cut of about 50% or greater.

7. The method of claim 1 wherein the low-dosage hydrate inhibitor additive is introduced in an amount such that the low-dosage hydrate inhibitor additive is present in the fluid in an amount from about 0.1% to about 10% volume based on a water cut of the fluid.

8. The method of claim 1 wherein the water is selected from the group consisting of: brine, deionized water, and any combination thereof.

9. A method comprising:
introducing a low-dosage hydrate inhibitor additive into a wellhead of a wellbore penetrating at least a portion of a subterranean formation, wherein the low-dosage hydrate inhibitor additive comprises at least one compound having the structural formula: 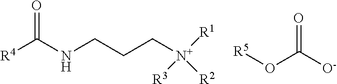
wherein each of R¹, R², and R³ is independently a C₁ to C₆ hydrocarbon chain,
wherein R⁴ is a C₁ to C₅₀ hydrocarbon chain, and
wherein R⁵ is a methyl or ethyl group; and
allowing the low-dosage hydrate inhibitor additive to contact a fluid in the wellbore.

10. The method of claim 9 wherein the fluid comprises at least one component selected from the group consisting of: water, a gas, a liquid hydrocarbon, and any combination thereof.

11. The method of claim 9 wherein the fluid comprises water and has a water cut of about 50% or greater.

12. The method of claim 11 wherein the water is selected from the group consisting of: brine, deionized water, and any combination thereof.

13. The method of claim 9 wherein the wellbore has a temperature from about 200° F. to about 350° F.

14. The method of claim 9 wherein the low-dosage hydrate inhibitor additive does not substantially degrade after about 7 days.

15. The method of claim 9 wherein the low-dosage hydrate inhibitor additive is introduced in an amount such that the low-dosage hydrate inhibitor additive is present in the fluid in an amount from about 0.1% to about 10% volume based on a water cut of the fluid.

16. A method comprising:
introducing a low-dosage hydrate inhibitor additive into a conduit containing a fluid, wherein the low-dosage hydrate inhibitor additive comprises at least one compound having the structural formula: 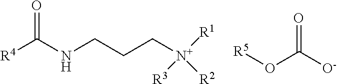
wherein each of R¹, R², and R³ is independently a C₁ to C₆ hydrocarbon chain,
wherein R⁴ is a C₁ to C₅₀ hydrocarbon chain, and
wherein R⁵ is a methyl or ethyl group.

17. The method of claim 16 wherein the fluid comprises at least one component selected from the group consisting of: water, a gas, a liquid hydrocarbon, and any combination thereof.

18. The method of claim 16 wherein the low-dosage hydrate inhibitor additive is introduced in an amount such that the low-dosage hydrate inhibitor additive is present in the fluid in an amount from about 0.1% to about 10% volume based on a water cut of the fluid.

19. The method of claim 16 wherein the conduit comprises a pipeline.

20. The method of claim 16 wherein the fluid comprises water and has a water cut of about 50% or greater.
